(19) United States
(12) Patent Application Publication
(10) Pub. No.: US 2023/0190328 A1
(43) Pub. Date: Jun. 22, 2023

(54) BALLOON CATHETER WITH SELECTIVE SCORING FUNCTION

(71) Applicant: C.R. BARD, INC., Franklin Lakes, NJ (US)

(72) Inventor: Matt Casiraro, Tempe, AZ (US)

(21) Appl. No.: 17/928,389

(22) PCT Filed: Jun. 12, 2020
(86) PCT No.: PCT/US2020/037402

§ 371 Date: Nov. 29, 2022

(51) Int. Cl. A61B 17/3207

(52) U.S. Cl. CPC A61B 17/320725; A61B 2017/320733; A61B 2017/22061

(57) ABSTRACT

A catheter (10) for introduction into a body vessel includes a shaft, a balloon (12) positioned at the distal end of the shaft, and at least one scoring wire (30) to score a vascular lesion. A distal end portion of the scoring wire is detachably connected to the catheter, such as by way of magnetic coupling (30a), thereby allowing for selective use of the catheter in a scoring or non-scoring configuration. The balloon expands when fluid is delivered to the balloon through an inflation lumen. This expansion pushes the scoring wire against the vascular lesion.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

Balloon dilatation catheters are used to treat lesions in vessels. However, difficulties are encountered in navigating tortuous anatomy and safely crossing very tight lesions. Moreover, some lesions are difficult to dilate using just a balloon, and require a focused force to dilate the lesion at safe inflation pressures.

U.S. Pat. No. 6,394,995 to Solar et al. describes a system used to provide enhanced force to treat a lesion. This system has a flexible advancement member with a tracking member slidable over a guidewire, and a balloon having a distal end attached to the tracking member. This type of system fixes the location of the scoring wires, which can limit flexibility in certain applications.

### SUMMARY OF THE INVENTION

An object of the disclosure is a balloon catheter that can be used either as a conventional, non-scoring balloon catheter or for providing scoring functionality, when such is desired.

In one aspect, the disclosure relates to a scoring balloon catheter comprising a shaft having a proximal end portion and a distal end portion. An inflatable balloon is mounted on the distal end portion of the shaft. At least one scoring wire includes a portion extending alongside the inflatable balloon. The shaft and the at least one scoring wire are adapted to form a magnetic coupling.

In one embodiment, the at least one scoring wire comprises a magnet for magnetically coupling with the shaft. The magnet may comprise a magnetic band attached to a distal end portion of the at least one scoring wire. Alternatively or additionally, the shaft may comprise a magnet for magnetically coupling with the at least one scoring wire. The magnet of the shaft may be associated with a tip portion thereof, and may comprise a magnetic band attached to the shaft.

In some embodiments, the balloon catheter includes a plurality of scoring wires, each magnetically coupled to the shaft. A proximal end of the scoring wire(s) may be adapted to move longitudinally relative to the shaft, and may also extend at least partially within the shaft.

According to a further aspect of the disclosure, a scoring balloon catheter comprises a shaft having a proximal end portion and a distal end portion , and an inflatable balloon mounted on the distal end portion of the shaft. At least one scoring wire includes a portion extending alongside the inflatable balloon. A magnetic coupling connects the at least one scoring wire to the shaft.

In one embodiment, the magnetic coupling is formed between a tip portion of the shaft and a distal end portion of the at least one scoring wire. The at least one scoring wire may comprise a magnet for forming the magnetic coupling. The shaft may comprise a magnet for forming the magnetic coupling, which magnet may be associated with a tip portion of the shaft, and/or may comprise a magnetic band attached to the shaft.

In some embodiments, a plurality of scoring wires each magnetically couple to the shaft. A proximal end of the at least one scoring wire may be adapted to move longitudinally relative to the shaft. The at least one scoring wire may also extend at least partially within the shaft.

According to a further aspect of the disclosure, a scoring balloon catheter includes an inflatable balloon for selective use in connection with at least one scoring wire including a portion extending alongside the inflatable balloon. A shaft includes a proximal end portion and a distal end portion supporting the balloon. The distal end portion of the shaft includes a magnet for forming a magnetic coupling with the at least one scoring wire. The magnet may be associated with a tip of the shaft, and may comprise a magnetic band attached to the shaft.

Still a further aspect of the disclosure relates to a scoring balloon catheter. The scoring balloon catheter comprises a shaft having a proximal end portion and a distal end portion . An inflatable balloon is mounted on the distal end portion of the shaft. At least one scoring wire includes a portion extending alongside the inflatable balloon and a magnet.

Yet another aspect of the disclosure pertains to a method of forming a scoring balloon catheter. The method comprises magnetically coupling a scoring wire running alongside an inflatable balloon to a shaft. The method may further comprise the step of locating a portion of the scoring wire within the shaft.

### BRIEF DESCRIPTION OF THE DRAWINGS

The above and further advantages of the present invention may be better understood by referring to the following description in conjunction with the accompanying drawings in which:

FIG. 1 is a perspective view of a balloon catheter.

FIG. 2 is a cutaway rear perspective view of the balloon portion of the balloon catheter of FIG. 1.

FIG. 2A is a cross-sectional view taken along line 2A-2A of FIG. 2.

FIG. 3 is an alternative embodiment of a balloon catheter.

FIG. 4 illustrates a scoring wire adapted to form a magnetic coupling with a distal end portion of a balloon catheter.

FIG. 5 illustrates one scoring wire attached to a balloon catheter via magnetic coupling.

FIG. 6 illustrates a balloon catheter with two magnetically connected scoring wires in use.

FIGS. 7 and 8 disclose an alternative embodiment.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The present disclosure is directed to systems and methods for treatment of a vessel. The principles and operation of systems and methods of the present invention may be better understood with reference to the drawings and accompanying descriptions.

The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

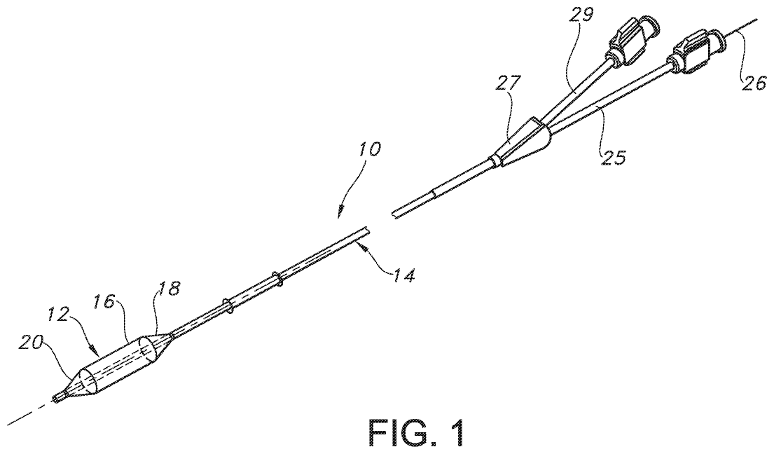

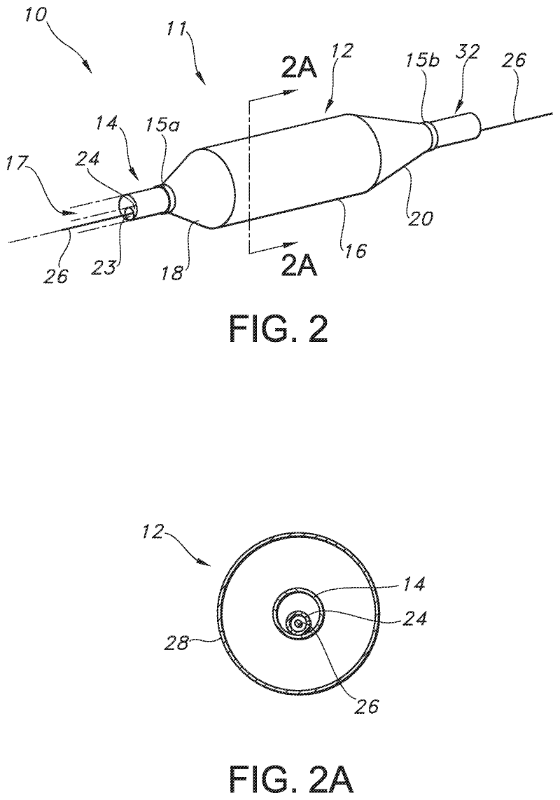

With reference to FIGS. 1, 2, and 2A, provided is a catheter 10 having a distal end portion 11 with a balloon 12 mounted on a catheter shaft 14. The balloon 12 has an intermediate portion 16, or “barrel,” and end portion s 18, 20. In one embodiment, the end portion s 18, 20 reduce in diameter to join the intermediate portion 16 to the catheter shaft 14 (and thus portion s 18, 20 are generally termed cones or cone portions). The balloon 12 is sealed at balloon ends (proximal end 15a and distal end 15b) on the cone portions 18, 20 to allow the inflation of the balloon 12 via one or more inflation lumens 17 extending within catheter shaft 14 and communicating with the interior of the balloon 12.

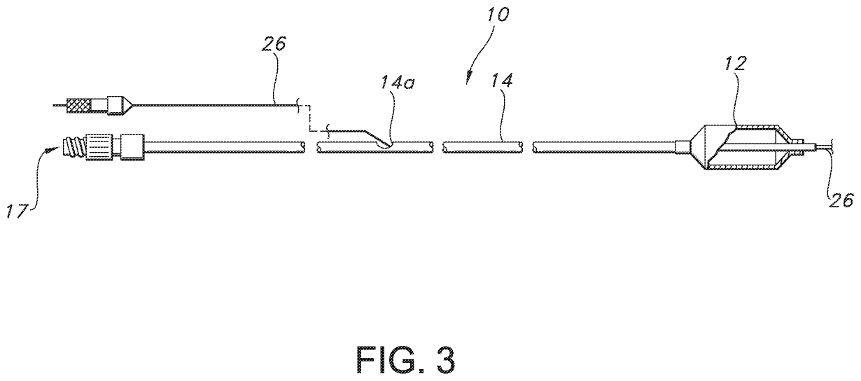

The catheter shaft 14 also includes an elongated, tubular shaft 24 forming a guidewire lumen 23 that directs the guidewire 26 through the catheter 10, and along the distal end portion of which the balloon 12 may be located. As illustrated in FIG. 2, this guidewire 26 may extend through the proximal end portion of the catheter 10 and a first port 25 of a connector or hub 27 into the lumen 23 to achieve an “over the wire” (OTW) arrangement, but could also be provided in a “rapid exchange” (RX) configuration (in which the guidewire 26 exits a lateral opening 14a closer to the distal end; see FIG. 3) or else is fed through the tip at a passage distally of the balloon 12 (“short” RX; not shown). A second port 29 may also be associated with catheter 10, such as by way of connector 27, for introducing a fluid (e.g., saline, a contrast agent, or both) into the interior compartment of the balloon 12 via the inflation lumen 17.

Inflatable balloon 12 may include a single or multi-layered balloon wall 28 forming the interior for receiving the inflation fluid. The balloon 12 may be a non-compliant balloon having a balloon wall 28 that maintains its size and shape in one or more directions when the balloon is inflated. The balloon 12 in such case also has a pre-determined surface area that remains constant during and after inflation, also has a pre-determined length and pre-determined diameter that each, or together, remain constant during and after inflation. However, the balloon 12 could be semi-compliant or compliant instead, depending on the particular use. The balloon 12 can have any of a variety of diameters ranging from 1.25-40 mm or 2.0-8.0 mm. In any of the embodiments set out herein, inflatable balloon 12 can have any of a variety of lengths such as 10-300 mm or 20-300 mm. Longer balloons may be particularly useful for treating peripheral lesions, which often have long diseased portions.

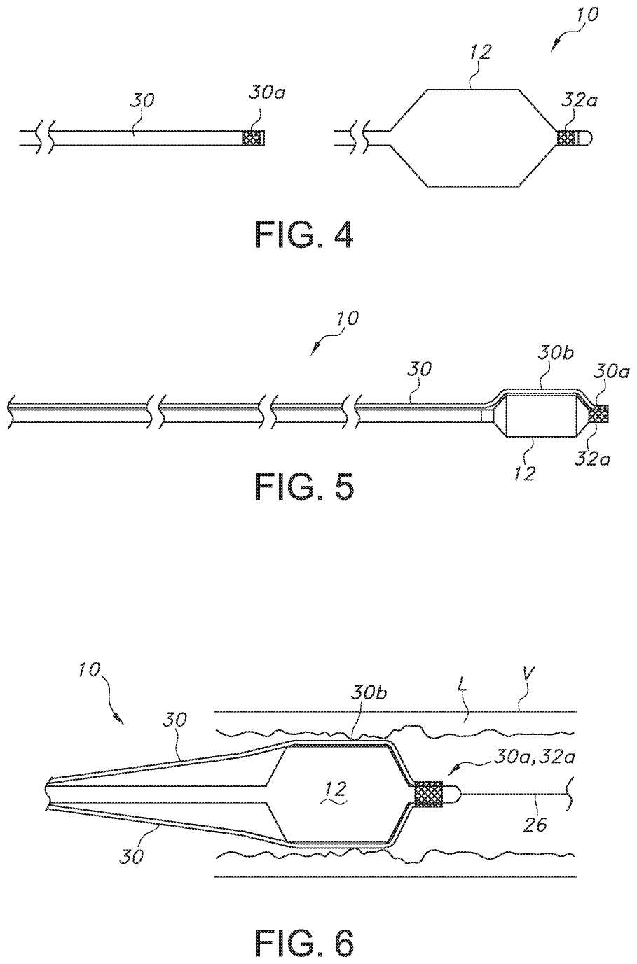

According to a first aspect of the disclosure, and with reference to FIGS. 4, 5, and 6, the catheter 10 may be provided with one or more scoring wires 30. The scoring wire 30 in this example is adapted for forming a magnetic coupling with the catheter 10, such as adjacent to tip portion 32 distal of the balloon 12. To form the magnetic coupling, the scoring wire 30 may be provided a magnetic or ferromagnetic portion, such as a magnet or magnetic band 30a at a distal end for establishing a detachable magnetic connection with the tip portion 32. Likewise, the tip portion 32 may be provided with a magnetic or ferromagnetic portion, such as a band 32a (adapted as necessary to form the magnetic coupling, such as for example 400 Series stainless steel in the case where the scoring wire 30 includes a magnetic band 30a, or visa-versa). In the case where stainless steel or like metals are used to form the magnetic coupling with a magnet, it should be appreciated that a radiopaque marker is provided as a result, which may help to identify the location of the scoring wire during an interventional procedure.

As illustrated in FIG. 5, the scoring wire 30 includes a portion 30b proximal of the end portion including band 30a (or otherwise adapted to couple with the shaft 14) that extends alongside the balloon 12. The remaining proximal portion of the scoring wire 30 may extend alongside the entire length of the catheter 10 to a proximal end. In this way, the scoring wire 30 may move in a longitudinal direction in response to inflation of the balloon 12.

As can be appreciated, the selective attachment achieved via magnetic coupling allows for the scoring wire 30 to be selectively used in connection with the catheter 10 or not (such as for completing percutaneous transluminal angioplasty (PTA) without a scoring function, in which case a scoring wire would not be used). In other words, the catheter 10 may be useful as a standard PTA catheter, or associated with one or more scoring wires, as desired to achieve a particular treatment effect.

In the situation where the band 32a comprises a magnet, a magnetic field external to the shaft 14 is created to form the magnetic coupling with the scoring wire 30.

In some embodiments the diameter of scoring wire 30 is between 0.003 inches and 0.040 inches, or 0.005 inches and 0.015 inches, 0.008 inches and 0.012 inches. In some embodiments, scoring wire 135 is 0.10 inches. Scoring wire 30 need not have a uniform diameter, and may have a larger diameter at a distal end as compared to a proximal end, or visa-versa. In some embodiments, scoring wire 30 comprises metal, metal alloy, polymer, and/or a shape memory material.

As can be understood from FIGS. 1, 2 and 6, when scoring functionality is desired, the scoring wire(s) 30 (two shown in this figure, but it could be only one or more than two) may be connected to the catheter 10. The physician then gains access to the vessel V opens the vessel, inserts a guidewire 26 into the vessel V, and advances the guidewire 26 through the patient's vasculature under ultrasound, magnetic resonance, fluoroscopic, or some other type of guidance. Once the physician places the guidewire 26 at a satisfactory site, the physician threads the proximal end of the guidewire 26 into the guidewire lumen 23.

With the scoring wire(s) in place and connected to catheter 10, the physician maneuvers the catheter 10 along the guidewire until inflatable balloon 12 reaches the desired position near the site of a lesion L. Typically, this position will allow at least one scoring wire to effectively engage the lesion. After that, the physician inflates inflatable balloon 12 until scoring wire(s) 30 firmly press into or crack the lesion L. Once lesion treatment with catheter 10 is complete, the physician deflates inflatable balloon 12, which allows scoring wire(s) 30 to relax away from the lesion L and from the wall of the vessel V.

Scoring wire(s) 30 may contacts the lesion as long as inflatable balloon 12 remains inflated. The inflation time corresponds to the times the physician chooses for scoring wire to contact the lesion. Inflation times may range from 5 seconds to 5 minutes. The nature of the lesion may determine the appropriate inflation time and inflation speed. Once the catheter 10 is removed from the vessel V, the scoring wire(s) 30 may be optionally removed for reuse.

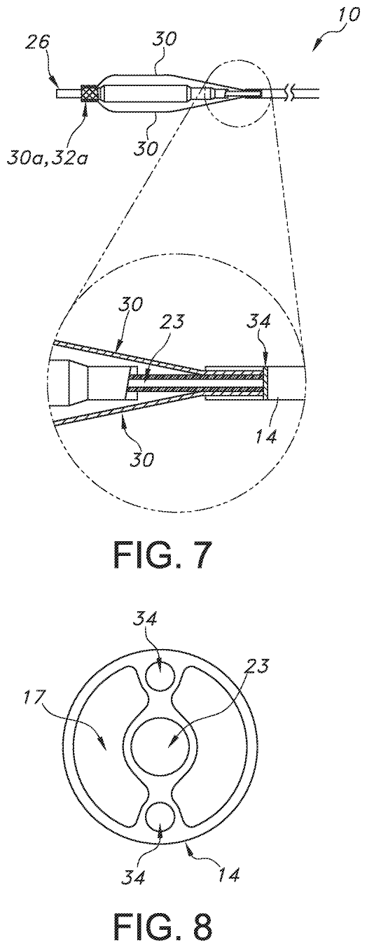

In the above version, the scoring wires 30 extend entirely external to the catheter 10, but could also be partially located within shaft 14. Specifically, with reference to FIGS. 7 and 8, a catheter 10 is illustrated with two scoring wires 30, each of which enters a lumen 34 formed in the shaft 14 (which as noted above and illustrated, may also include a guidewire lumen 23 and one or more inflation lumens 17). Each scoring wire 30 is magnetically coupled at a distal end portion, such as with a magnet or magnetic band 30a, to part or all of the tip portion 32, which is attractive thereto (e.g., via magnet or ferromagnetism), to form a detachable connection. A proximal end of each scoring wire 30 may also be connected to the shaft 14 for relative movement, such as for example by a spring, to allow for longitudinal expansion when the balloon 12 is inflated.

Summarizing, this disclosure may be considered to relate to the following items:
1. A scoring balloon catheter, comprising:

a shaft having a proximal end portion and a distal end portion;

an inflatable balloon mounted on the distal end portion of the shaft; and

at least one scoring wire including a portion extending alongside the inflatable balloon;

wherein the scoring balloon catheter, particularly the shaft and the at least one scoring wire, is/are configured to form a magnetic coupling (between the shaft and the at least one scoring wire).
2. The scoring balloon catheter of item 1, wherein the at least one scoring wire comprises a magnet for magnetically coupling with the shaft.
3. The scoring balloon catheter of item 2, wherein the magnet of the scoring wire comprises a magnetic band attached to a distal end portion of the at least one scoring wire.
4. The scoring balloon catheter of any of items 1-3, wherein the shaft comprises a magnet for magnetically coupling with the at least one scoring wire.
5. The scoring balloon catheter of item 4, wherein the magnet of the shaft is associated with a tip of the shaft.
6. The scoring balloon catheter of item 4 or 5, wherein the magnet of the shaft comprises a magnetic band attached to the shaft.
7. The scoring balloon catheter of any of items 1-6, further including a plurality of scoring wires, each magnetically coupled to the shaft.
8. The scoring balloon catheter of any of items 1-7, wherein a proximal end of the at least one scoring wire is adapted to move longitudinally relative to the shaft.
9. The scoring balloon catheter of any of items 1-8, wherein a portion of the at least one scoring wire is within the shaft.
10. A scoring balloon catheter, comprising:

a shaft having a proximal end portion and a distal end portion;

an inflatable balloon mounted on the distal end portion of the shaft;

at least one scoring wire including a portion extending alongside the inflatable balloon; and

a magnetic coupling (optionally detachably) connecting (configured to connect) the at least one scoring wire to the shaft. (The at least one scoring wire and/or the shaft may comprise a magnetic coupling, the magnetic coupling configured to, optionally detachably, connect the least one scoring wire to the shaft.)
11. The scoring balloon catheter of item 10, wherein the magnetic coupling is formed between a tip portion of the shaft and a distal end portion of the at least one scoring wire.
12. The scoring balloon catheter of item 10 or item 11, wherein the at least one scoring wire comprises a magnet for forming the magnetic coupling.
13. The scoring balloon catheter of any of items 9-12, wherein the shaft comprises a magnet for forming the magnetic coupling.
14. The scoring balloon catheter of item 13, wherein the magnet of the shaft is associated with a tip of the shaft.
15. The scoring balloon catheter of item 12 or item 13, wherein the magnet of the shaft comprises a magnetic band attached to the shaft.
16. The scoring balloon catheter of any of items 10-15, further including a plurality of scoring wires, each magnetically coupled to the shaft.
17. The scoring balloon catheter of any of items 10-15, wherein a proximal end of the at least one scoring wire is adapted to move longitudinally relative to the shaft.
18. The scoring balloon catheter of any of items 10-15, wherein a portion of the at least one scoring wire is within the shaft.
19. A balloon catheter for use in connection with at least one scoring wire, comprising:

an inflatable balloon; and

a shaft having a proximal end portion and a distal end portion supporting the balloon, the distal end portion including a magnet for producing a magnetic field external to the shaft for forming a magnetic coupling with the at least one scoring wire.
20. The scoring balloon catheter of item 19, wherein the magnet is associated with a tip of the shaft.
21. The scoring balloon catheter of item 19 or 20, wherein the magnet comprises a magnetic band attached to the shaft, such as along an outer surface thereof.
22. A scoring balloon catheter, comprising:

a shaft having a proximal end portion and a distal end portion;

an inflatable balloon mounted on the distal end portion of the shaft; and

at least one scoring wire including a portion extending alongside the inflatable balloon and a magnet.
23. A method of forming a scoring balloon catheter, comprising:

magnetically coupling a scoring wire running alongside an inflatable balloon to a shaft.
24. The method of item 23, further including the step of locating a portion of the scoring wire within the shaft.

Although the invention has been described in conjunction with specific embodiments, many alternatives, modifications, and variations will be apparent to those skilled in the art. Accordingly, it embraces all such alternatives, modifications, and variations that fall within the appended claims' spirit and scope. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. A scoring balloon catheter, comprising:
a shaft having a proximal end portion and a distal end portion;
an inflatable balloon mounted on the distal end portion of the shaft; and
at least one scoring wire including a portion extending alongside the inflatable balloon;
wherein the shaft and the at least one scoring wire are adapted to form a magnetic coupling.

2. The scoring balloon catheter of claim 1, wherein the at least one scoring wire comprises a magnet for magnetically coupling with the shaft.

3. The scoring balloon catheter of claim 2, wherein the magnet comprises a magnetic band attached to a distal end portion of the at least one scoring wire.

4. The scoring balloon catheter of claim 1, wherein the shaft comprises a magnet for magnetically coupling with the at least one scoring wire.

5. The scoring balloon catheter of claim 4, wherein the magnet is associated with a tip of the shaft.

6. The scoring balloon catheter of claim 4, wherein the magnet comprises a magnetic band attached to the shaft.

7. The scoring balloon catheter of claim 1, further including a plurality of scoring wires, each magnetically coupled to the shaft.

8. The scoring balloon catheter of claim 1, wherein a proximal end of the at least one scoring wire is adapted to move longitudinally relative to the shaft.

9. The scoring balloon catheter of claim 1, wherein a portion of the at least one scoring wire is within the shaft.

10. A scoring balloon catheter, comprising:
a shaft having a proximal end portion and a distal end portion;
an inflatable balloon mounted on the distal end portion of the shaft;
at least one scoring wire including a portion extending alongside the inflatable balloon; and
a magnetic coupling connecting the at least one scoring wire to the shaft.

11. The scoring balloon catheter of claim 10, wherein the magnetic coupling is formed between a tip portion of the shaft and a distal end portion of the at least one scoring wire.

12. The scoring balloon catheter of claim 10, wherein the at least one scoring wire comprises a magnet for forming the magnetic coupling.

13. The scoring balloon catheter of claim 10, wherein the shaft comprises a magnet for forming the magnetic coupling.

14. The scoring balloon catheter of claim 13, wherein the magnet is associated with a tip of the shaft.

15. The scoring balloon catheter of claim 13, wherein the magnet comprises a magnetic band attached to the shaft.

16. The scoring balloon catheter of claim 10, further including a plurality of scoring wires, each magnetically coupled to the shaft.

17. The scoring balloon catheter of claim 10, wherein a proximal end of the at least one scoring wire is adapted to move longitudinally relative to the shaft.

18. The scoring balloon catheter of claim 10, wherein a portion of the at least one scoring wire is within the shaft.

19. A balloon catheter for selective use in connection with at least one scoring wire, comprising:
an inflatable balloon; and
a shaft having a proximal end portion and a distal end portion supporting the balloon, the distal end portion of the shaft including a magnet for producing an external magnetic field for forming a magnetic coupling with the at least one scoring wire.

20. The scoring balloon catheter of claim 19, wherein the magnet is associated with a tip of the shaft.

21. The scoring balloon catheter of claim 19, wherein the magnet comprises a magnetic band attached to the shaft.

22.-24. (canceled)
